(19) United States
(12) Patent Application Publication
(10) Pub. No.: US 2004/0191303 A1
(43) Pub. Date: Sep. 30, 2004

(54) Use of selectin-binding pregnancy proteins, liposomes, native mucin fragments and mimetic compounds for the treatment and prophylaxis inflammatory diseases, for preventing metastatic spread and for the prophylaxis of tumour diseases

(76) Inventors: Renate Stahn, Berlin (DE); Steffen Goletz, Berlin (DE); Udo Jeschke, Rostock (DE); Iduna Fichtner, Berlin (DE); Reinhard Zeisig, Berlin (DE)

Correspondence Address: CHRISTINA HILDEBRAND, NORRIS, MCLAUGHLIN & MARCUS, 220 EAST 42ND STREET - 30TH FLOOR, NEW YORK, NY, 10017, US

(21) Appl. No.: 10/416,061

(22) PCT Filed: Nov. 7, 2001
(86) PCT No.: PCT/EP01/12874

(30) Foreign Application Priority Data
Nov. 7, 2000 (DE) 100-56-1 3 6.5

(51) Int. Cl. A61K 39/395; A61K 9/127

(52) U.S. Cl. 424/450; 424/130.1

(57) ABSTRACT

A selectin-binding active agents in the form of gestation proteins or fragments thereof, of liposomes which include Ca-binding compounds, of mucin fragments obtained or derived from native sources, or of mimicry compounds which imitate sialylated Lewis type carbohydrate structures (sLe), or combinations thereof, in the treatment and prophylaxis of diseases, in the course of which inflammatory processes are involved, such as autoimmune diseases, transplantations and arteriosclerosis. Inflammatory diseases in the meaning of the invention can be those of infectious or non-infectious nature. The active agents provide partial or complete prevention of tumor metastasizing, wherein administration of the active agents can be prophylactic, or can be effected in association with e.g. operative removal of a primary tumor or during a biopsy. The above active agents are used in the prophylaxis of tumor diseases.

### DESCRIPTION

The invention relates to the use of selectin-binding active agents in the form of gestation proteins or fragments thereof, of liposomes which include Ca-binding compounds, of mucin fragments obtained or derived from natural sources, or of mimicry compounds which imitate sialylated Lewis type carbohydrate structures (sLe), or combinations thereof, in the treatment and prophylaxis of diseases, in the course of which inflammatory processes are involved, such as autoimmune diseases, transplantations and arteriosclerosis. Inflammatory diseases in the meaning of the invention can be those of infectious or non-infectious nature.

The invention is also directed to the use of the above active agents in partial or complete prevention of tumor metastasizing, wherein administration of the active agents can be prophylactic, or can be effected in association with e.g. operative removal of a primary tumor or during a biopsy. In addition, the invention is also directed to the use of the above active agents in the prophylaxis of tumor diseases.

The use of the active agents according to the invention relates to both human medicine and veterinary medicine.

The invention also relates to pharmaceutical agents in accordance with claims 10-18, which include these active agents.

As is known from the literature, peptides and liposomes with sialylated Lewis x- or sialylated Lewis a-carbohydrate ligands (sLe^{x} or sLe^{a}) inhibit adhesion of leukocytes or tumor cells to E- or P-selectins (surface proteins expressed by activated vascular endothelial cells) [cf., e.g. Sh. A. DeFrees et al., J. Am. Chem. Soc. 118 (1996), 6101-6104; R. Stahn et al., Glycobiology Vol. 8, No. 4 (1998), 311-319]. Lewis carbohydrate structures bind to the lectin domain in these selecting, thereby inhibiting cell adhesion from the bloodstream. As is also familiar, more efficient blockage of selectins can be achieved e.g. with di- and trivalent sLe^{x} peptides [(sLe^{x})₂ peptides and (sLe^{x})₃ peptides] and with sLe^{x} liposomes having a plurality of Lewis carbohydrate residues as component of the membrane. Such multivalency of the carbohydrate-selectin bonds results in improved inhibition of the adhesion of cells to the selectin(s).

The literature also describes that mucins bearing sLe^{a} or sLe^{x} bind to E-selectin and inhibit leukocyte adhesion or adhesion of tumor cells to E-selectin [K. Zang et al., Tumor Biology 18 (1997), 175-187; T. Sawada et al., Int. J. Cancer 57 (1994), 901-907]. Mucins are high-molecular weight glycoproteins.

The object of the present invention was to find alternative compounds which inhibit adhesion of cells from the bloodstream to activated endothelial cell tissue of the blood vessels, or to find compounds which exhibit a more efficient inhibitory effect by binding to activated endothelial cell tissue with higher specificity and affinity compared to prior art inhibitors described so far. The compounds should be suitable as active agents in the prophylaxis and therapy of inflammatory diseases and tumor diseases.

Surprisingly, human or animal gestation proteins were found to be extremely efficient inhibitors of adhesion of cells from the bloodstream to activated vascular endothelium. This function is new and will be referred to hereinafter as activity in the meaning of the invention. These proteins bind to selectins with specificity and high affinity.

In the meaning of the invention, those proteins formed by the placenta during pregnancy are used as gestation proteins. In particular, such proteins are human gestation proteins, preferably gonadotropic hormones such as FSH (follicle-stimulating hormone), LH (luteinizing hormone) hCG (human chorionic gonadotropin), or α-fetoprotein, transferrin, glycodelins, particularly glycodelin A (pp14), or fragments thereof. According to the invention, it is possible to use proteins and glycoproteins isolated from human or animal amniotic fluid or serum or urine, as well as proteins or partial peptides produced by synthesis, which may have appropriate glycosylations and exhibit the same properties as native proteins formed by the placenta during pregnancy. The functional activity of the gestation proteins in the meaning of the invention is attributed to a specific glycosylation hitherto unknown in these proteins. The examples describe the gestation protein pp14 and demonstrate that pp14 from urine, serum and amniotic fluid from pregnant women can be used and has comparable activity in the meaning of the invention.

Surprisingly, other gestation proteins obtained from various sources have varying activities in the meaning of the invention [as demonstrated in Examples 1, 2 and 4 for hCG, transferrin and α-fetoprotein (Table 1 and Table in Example 4)]. Thus, hCG and α-fetoprotein from the serum of pregnant women and from the amniotic fluid have high, respectively highest, activity in the meaning of the invention, while urinary hCG from pregnant women is not suitable due to low activity in the meaning of the invention. Transferrin recovered from the amniotic fluid is the only one which is active in the meaning of the invention.

The proteins can also be obtained from pregnancy-associated cell cultures derived from the placenta, such as trophoblast cultures, non-modified or modified by accumulation, stimulation using suitable molecules and/or transfection of suitable genes expressing the desired gestation proteins or parts thereof, including the suitable glycosylations. For example, hCG isolated from trophoblast cell cultures/cell lines is suitable for use according to the invention.

The gestation proteins used according to the invention may also be coupled to suitable biological or chemical carrier molecules or particles, such as proteins, bacteriophages or liposomes, preferably liposomes containing Ca-complexing compounds.

In another embodiment of the invention, liposomes which bind to selectin, include Ca-binding compounds, especially EDTA, and bear sialylated Lewis type carbohydrate structures in the form of glycolipids, glycoproteins or glycopeptides as components of the liposomal membrane are used for efficient inhibition of adhesion of cells from the bloodstream to activated endothelial cell tissue of the blood vessels. The liposomes used according to the invention are preferably present in the form of single- or multilayered vesicles and consist of a base lipid, preferably phosphatidylcholine, and an anchor lipid, preferably phosphatidylethanolamine, and include a Ca-binding or complexing compound, e.g. ethylenediaminetetraacetic acid (EDTA), as additional active component. The carbohydrate ligand is bound to the anchor lipid e.g. via a spacer which can be a polyethylene glycol chain, a peptide or an alkyl group. To this end, sLe^{x}-polyethylene glycol(2000)-distearylphosphoethanolamine is preferably used. Charge carriers such as diacetylphosphate and membrane stabilizers such as cholesterol are possible as additional membrane components.

The preparation of such glycoliposomes is well-known to those skilled in the art.

According to the invention, it is also possible to use liposomes which include Ca-binding compounds and bear antibodies, antibody fragments, peptides or other proteins or fragments thereof, e.g. gestation proteins, which bind to selectin. Likewise, it is possible to use liposomes which include Ca-binding compounds and bear mimicry compounds imitating the sLe structures.

Surprisingly, the liposomes of the invention exhibit considerably higher inhibitory activity compared to the “empty” glycoliposomes described in the literature. Thus, for example, the EDTA glycoliposomes in accordance with Example 4 show an inhibitory effect on tumor cell binding increased by many times compared to glycoliposomes of same composition, but with no entrapped EDTA.

In another embodiment of the invention, low-molecular weight fragments of mucins from natural sources, e.g. from autologous body fluids or cell cultures, are used to inhibit adhesion of cells from the bloodstream to activated endothelial cells.

Mucins are high-molecular weight glycoproteins capable of triggering immunoreactions, which is why their clinical use as adhesion blockers is problematic. Such problems can be avoided by using the inventive low-molecular weight fragments which have sialylated Lewis type carbohydrate structures and are prepared from natural mucins. Surprisingly, the low-molecular weight fragments exhibit improved inhibition compared to the mucins described in the literature.

For example, the mucin fragments according to the invention are produced by enzymatic degradation. Alternatively, the mucin fragments of the invention can also be produced by synthesis.

The production of such mucin fragments by enzymatic degradation is well-known to those skilled in the art, which also applies to the synthesis of these fragments, including appropriate glycosylations.

According to the invention, the mucins may also be coupled to suitable biological or chemical carrier molecules or particles, such as proteins, bacteriophages or liposomes, preferably liposomes including Ca-complexing compounds.

In yet another embodiment, the object of the invention is accomplished by using compounds which imitate said Lewis type carbohydrate structures (so-called mimicry compounds) and bind to selectins with high specificity and affinity, which compounds are obtained with the aid of molecules recognizing the Lewis type carbohydrate structure. For example, such compounds can be linear or cyclic peptides, antibodies or anti-body fragments, or other protein structures such as protein scaffolds with variable sections, which have an effect similar to that of antibody fragments.

Mimicry compounds in the form of mimicry peptides, antibodies, antibody fragments, proteins with variable sections, are produced by

a) preparing or selecting monoclonal antibodies by means of hybridoma techniques using substances specifically recognizing Lewis type carbohydrates (e.g. antibodies or lectins which are not selecting), which antibodies bind the carbohydratebinding regions of these substances, thereby imitating Lewis type carbohydrates;

b) preparing or selecting recombinant antibody fragments such as single-chain antibody fragments (scFv) or Fab fragments from genomic, hybrid, semisynthetic or synthetic antibody gene libraries and from gene libraries of immunized or non-immunized donors by means of phage display techniques or ribosome display techniques using substances specifically recognizing Lewis type carbohydrates (e.g. anti-bodies or lectins which are not selecting), which fragments bind the carbohydrate-binding regions of these substances, thereby imitating Lewis type carbohydrates;

c) preparing or selecting linear or cyclic peptides from synthetic peptide gene libraries by means of phage display techniques or ribosome display techniques using substances specifically recognizing Lewis type carbohydrates (e.g. antibodies or lectins), which peptides bind the carbohydrate-binding regions of these substances, thereby imitating Lewis type carbohydrates;

d) preparing or selecting proteins from protein gene libraries, which represent proteins including synthetic or semisynthetic variable sections, e.g. by means of phage display techniques or ribosome display techniques using substances specifically recognizing Lewis type carbohydrates (e.g. antibodies or lectins which are not selecting), which proteins bind the carbohydrate-binding regions of these substances, thereby imitating Lewis type carbohydrates, and producing a structure corresponding to the antibodies, proteins or peptides according to a-d, or corresponding to suitable partial peptides or derived peptides, e.g. by cyclization, mutations, in the form of inverse or retroinverse peptides or repetitive constructs according to per se known methods.

According to the invention, mimicry compounds preferably are produced using an sLe^{x}- or sLe^{a}-specific antibody imitating the sLe^{x} or sLe^{a} carbohydrate as a mimicry molecule.

To date, the mimicry compounds of the invention have not been described as substances per se, neither is there a description as to the inventive use thereof. They effectively prevent binding of tumor cells and leukocytes to selectins and can therefore be used in the prophylaxis or therapy of inflammatory diseases and tumor diseases. In a particularly preferred embodiment, the mimicry compounds for use according to the invention are coupled to liposomes, preferably such liposomes containing Ca-complexing compounds such as EDTA.

As illustrated above, the mimicry compounds can be linear or cyclic peptides—the latter frequently having higher serum stability—or, alternatively, inverse or retroinverse peptides which are relatively stable. Antibodies or antibody fragments, single-chain (scFv) or Fab antibody fragments are also used according to the invention, human antibody fragments being highly advantageous in that they normally do not induce any immune reaction to mouse or other foreign antibodies which would bind the antibodies, thereby neutralizing the antibodies after a short period of time, which is why human antibody fragments can also be used repeatedly—a fact which is advantageous in inhibiting inflammatory reactions and preventing or reducing formation of metastases. Other proteins also find use, preferably those including a basic backbone (scaffold) of a human protein in combination with variable sections (e.g. affibodies) essentially responsible for molecular mimicry.

One way of obtaining these molecules is selecting the molecules with the aid of phage display techniques by using an anti-sLe^{x} antibody as antigen and isolating the molecules binding the carbohydrate-specific binding site of the antibody from the corresponding libraries:

In addition to phage display techniques, ribosome display or comparable techniques are suitable in obtaining said mimicry molecules. Protein-based mimicry molecules can also be constructed with the aid of molecular modelling and produced on the recombinant route using molecular-biological methods. Mimicry molecules not based on proteins can also be obtained using a combination of chemical methods and/or molecular modelling.

For example, the libraries are peptide libraries representing linear or cyclic peptides; antibody libraries produced by synthetic, semisynthetic means, or from human material from healthy donors or patients; libraries representing a scaffold protein with randomized variable regions, such as affibodies.

Apart from sLe^{x}-specific mouse antibodies, it is also possible to use lectins, other antibodies or antibody fragments of human or animal origin, which recognize sLe^{x}, sLe^{a} or other Lewis type carbohydrates responsible for adhesion of tumor cells or leukocytes to the activated endothelium in the meaning of the present patent, in the production of mimicry structures.

Production in selections can be effected by means of specific elution, using a large excess of appropriate carbohydrates, with the advantage of reducing the operating time. However, to isolate the mimicry molecules of highest affinity, such a specific elution advantageously is omitted.

The selections of mimicry peptides and human scFv antibody fragments imitating sLe^{x} are illustrated in more detail in the examples.

Mimicry molecules involve several advantages:

the production of carbohydrates is very costly and complex; in contrast, mimicry peptides lacking a carbohydrate modification can be produced more rapidly and more favorably by synthetic or biological means, e.g. by means of molecular-biological methods using coupling to bacteriophages; antibody fragments and other proteins can be produced more rapidly and more favorably in bacteria or in animal cells on the recombinant route.

Mimicry molecules may have higher affinity to selectins. Hence, their inhibition potential is higher.

To further increase the affinity, multimeric mimicry entities in the form of molecules or particles are created: for example, by multiple coupling of the mimicry molecules to carrier proteins such as HSA; multiple expression of mimicry molecules as fusion proteins with bacterial coat proteins on bacteriophages; by coupling of mimicry molecules to lipids and incorporation in liposomes.

In the desired inhibition of cell adhesion in in vitro tests analogous to those described above, these molecules or particles with multiple mimicry molecules are superior to the original monomeric Lewis type carbohydrates by several orders of magnitude.

According to the invention, the mimicry compounds may also be coupled to suitable biological or chemical carrier molecules or particles such as proteins, bacteriophages or liposomes, preferably liposomes containing Ca-complexing compounds.

In another embodiment of the invention, selectin-specific antibodies or antibody fragments coupled to liposomes, preferably to liposomes containing Ca-complexing compounds such as EDTA, are employed with advantage to inhibit adhesion of cells from the bloodstream to activated vascular endothelium. Such antibodies are well-known. For example, the monoclonal, commercially available BBA2 antibody from R & D Systems can be used as antibody. In addition, the production of selectin-specific antibodies is not problematic to those skilled in the art.

In analogy, inhibition of cell adhesion can also be achieved by peptides or proteins which are coupled to liposomes and, at the same time, contain Ca-complexing compounds.

This embodiment involves the following advantages:

Multimerizing of bonds, thereby increasing the affinity owing to the avidity effect which leads to an inhibition of tumor cells expressing the Lewis type carbohydrates or of leukocytes to the activated endothelial cells which is enhanced by many times compared to single antibodies.

Human scFv or Fab, which have been isolated from the antibody libraries using the above-described selecting techniques and which, in principle, are monomeric antibody fragments, can be used directly. This is advantageous in that immune reaction to human antibody fragments is reduced.

Integration in the liposomal membrane. Owing to the lateral mobility of bondable ligands in the membrane, adjustment to the arrangement of selectins for effective binding is possible. In contrast, when coupling ligands to a rigid backbone (e.g., a rigid protein structure), steric hindrance may occur, possibly giving rise to reduced effectiveness of inhibition.

Owing to the inhibition of cell adhesion as described, the compounds described above are excellently suited as active agents in the prophylaxis and therapy of diseases, in the course of which inflammatory processes are involved. The compounds can be used alone or in synergistic combinations. Obviously, the effect of the compounds according to the invention can be further improved by suitable formulations, e.g. by adding immunostimulant or immune-inhibiting compounds such as lymphokines, cytokines, chemokines, or adjuvants.

The pharmaceuticals based on the above active substances are produced according to conventional methods well-known in the galenic technology using pharmaceutically conventional adjuvants.

In particular, the compounds of the invention find use in

a) prophylaxis to prevent or reduce metastasizing in high-risk situations, e.g. operation of tumors or biopsies in the event of suspected tumor;

b) prophylaxis to prevent or reduce metastasizing in the event of suspected tumor disease;

c) prophylaxis to prevent or reduce metastasizing in the event of a tumor disease;

d) prophylaxis to prevent or reduce metastasizing in the event of surgery associated with minimal residual tumor disease;

e) treatment of autoimmune diseases;

f) reduction of tissue lesions associated with surgery, transplantation, ischemia, and reperfusion;

g) treatment of diseases at an early stage of pregnancy;

h) reduction of atherosclerotic vascular changes, e.g. restenosis.

Without intending to be limiting, the invention will be illustrated in more detail with reference to the following examples.

### EXAMPLE 1

Isolation of amniotic hCG and testing the ability of inhibiting cell adhesion using the example of inhibition of binding of HepG2 hepatoma cells to E-selectin and to activated endothelial cells from veins of human umbilical cords (HUVEC).

### EXAMPLE 1a

### Isolation of hCG from Amniotic Fluid

The hCG is formed by syncytiotrophoblasts, following implantation of the fertilized ovum, and secreted into the blood circulation and amniotic fluid of pregnant women. Amniotic fluid samples from chromosomal analysis (500 ml) is dialyzed against PBS. 1 mg of mouse mAb rabbit-anti-human chorionic gonadotropin, DAKO (rabbit-anti-human chorionic gonadotropin), directed against the 13-subunit of hCG, is bound to the corresponding amount of CNBr Sepharose. The anti-hCG Sepharose is filled into a chromatographic column used for immunoadsorption of hCG. The hCG is isolated using 100 mM citrate buffer and further purified by means of FPLC using anion exchange chromatography on Resource Q. hCG consists of one α- and one β-subunit; in addition to intact hCG, the trophoblasts also form free α- and β-chains. The free β-chain accounts for about 2-3% of the intact hCG, reaching its maximum in the ₁₀^{th} week of gestation, as does the latter. In contrast, the free α-chain continuously increases during gestation, reaching its maximum in the 3^{rd} trimenon. Using the immunoadsorption column directed against the β-chain of hCG, the complete molecule and free β-chains are isolated in this way. Free α-chains are isolated using a second antibody column loaded with monoclonal antibodies directed against the α-chain of hCG. Using gel filtration on Superdex 75, the free β-chains are separated from the intact complete molecule and isolated. The purity of the preparation is checked using SDS-PAGE and silver staining.

### Example 1b

### Performing the Adhesion Inhibition Test

The adhesion test described below is used in vitro to test the active substances. To this end, either recombinant E-selectin is immobilized on 96-well titer plates, or the expression of E-selectin on activated endothelial cells from veins of human umbilical cords (HUVEC) is induced by stimulation with cytokines. Culturing of HUVEC endothelial cells is also performed in 96-well microtiter plates.

E-selectin (R & D Systems) (5 μg/ml) is immobilized by incubating at 4° C. over-night. The plates are washed with a calcium-containing phosphate buffer (Ca-PBS) and blocked with 1% bovine serum albumin (BSA) for 1 hour at room temperature. Amniotic hCG (0.005-1 nmol/well) is added and pre-incubated for 30 minutes at room temperature. Thereafter, 1×10^{5 51}Cr-labelled HepG2 hepatoma cells adhering to E-selectin are added, and this is incubated for another 30 minutes at room temperature. Subsequently, the unbound cells are removed by washing 3 times with Ca-PBS, and the bound cells, following lysis with 0.1 N sodium hydroxide solution, are quantified by means of radioactivity measurement. As controls, the carbohydrate-binding domain of E-selectin-blocking monoclonal antibody BBA2 (R & D Systems), EDTA to remove Ca from the test system, and the monovalent tetrasaccharide sialyl-Lewis x (sLe^{x}) binding selectively but with low affinity are used and carried along in parallel in the test series.

HUVECs are isolated from veins of fresh human umbilical cords, used up to the 3^{rd} passage, and cultured to confluence in 96-well titer plates for testing. Expression on HUVEC is induced by adding 0.2 ng of IL-1β per well, reaching its maximum after 4 hours. At this point, an adhesion test/adhesion inhibition test analogous to the one for immobilized E-selectin is performed.

Table 1 shows the efficacy of hCG isolated from various sources in both test systems. The IC₅₀ values (concentration of inhibiting agent for 50% inhibition of adhesion) are indicated.

TABLE 1

| | Immobilized | |
|---|---|---|
| | E-selectin | Activated HUVEC |
| Inhibiting agent | IC₅₀ [M] | IC₅₀ [M] |
| sLe^{x} | 1.5 × 10⁻³ | 2.3 × 10⁻³ |
| mAb BBA2 | 2 × 10⁻⁹ | 3 × 10⁻⁹ |
| Urinary hCG | None | None |
| Amniotic hCG | 6.2 × 10⁻⁸ | 1.5 × 10⁻⁷ |
| Serum hCG | 3.4 × 10⁻⁷ | 1.8 × 10⁻⁷ |
| Jeg3 hCG | 1.4 × 10−7 | 1 × 10⁻⁷ |
| (from trophoblast cell | | |
| line Jeg3) | | |

Clearly, hCG from amnion, serum, and trophoblast cultures, but not hCG from urine, is at least 10⁴ times more effective than sLe^{x}.

### EXAMPLE 2

### Isolation of hCG from Serum and Testing in Analogy to Example 1b

During gestation, hCG is continuously secreted into the serum, reaching its maximum in the first trimenon. 500 ml of pooled serum from pregnant women in the first trimenon is dialyzed 2 times against H₂O and subsequently against 20 mM NaH₂PO₄. Serum hCG is isolated in analogy to the isolation of amniotic hCG.

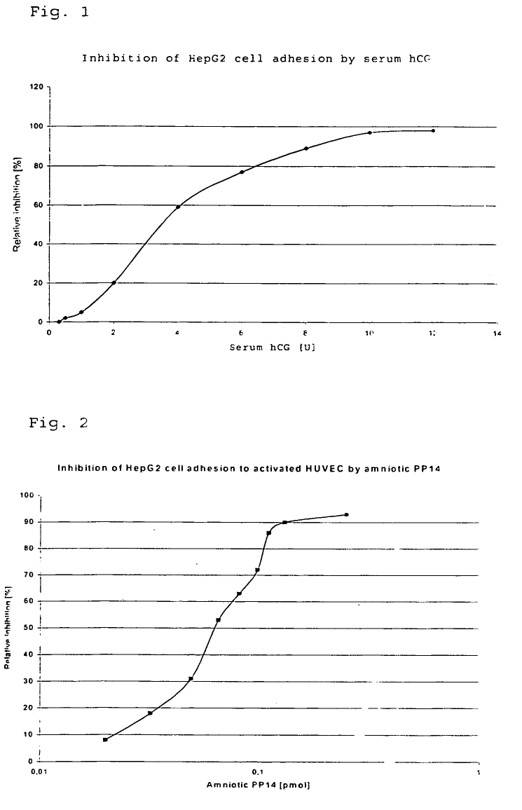

In an adhesion inhibition test following Example 1b, an effect about 3 times less compared to amniotic hCG, but an increase by 10⁴ times over the monovalent tetrasaccharide sLe^{x} is measured. FIG. 1 shows the inhibition of HepG2 cell adhesion as a function of serum hCG concentration.

### EXAMPLE 3

### Isolation of Glycodelin A (Amniotic PP14) and Testing in Analogy to Example 1b

The pre-purification of glycodelin A from amniotic fluid essentially proceeds according to a protocol wherein pooled amniotic fluid samples are dialyzed against water and subsequently against 50 mM NH₄HCO₃. This product is separated by chromatography on a DEAE-Sepharose column. The fraction including glycodelin A is further purified on a Superdex 75 column and subsequently on an Octyl-Sepharose column. Following this step of hydrophobic interaction chromatography on Octyl-Sepharose, the glycodelin A is purified on a Resource-Phe column using an isopropanol/phosphate buffer mixture as solvent.

Alternatively, glycodelin A can be purified using immunoadsorption chromatography. To this end, 1 mg of anti-glycodelin A mAb (mouse-anti-human glycodelin, DNA Diagnostik Nord GmbH) is bound to CNBr-Sepharose. The material is filled into a 5 ml chromatographic column. The immunoadsorption column thus produced is loaded with 500 ml of amniotic fluid dialyzed against 20 mM Na₂HPO₄ (pH 7.0). Glycodelin A is eluted with 100 mM citrate buffer. The purity of the preparation is checked using SDS-PAGE and silver staining.

In the adhesion inhibition test in analogy to Example 1b, an effectiveness comparable to amniotic hCG is measured. This activity is comparable to pp14 from urine, serum and amniotic fluid of pregnant women. FIG. 2 shows the inhibition of HepG2 cell adhesion to activated HUVEC as a function of glycodelin A concentration.

### EXAMPLE 4

### Testing of Gestation Proteins from Various Sources as Adhesion Blocker

| Protein | Adhesion blocking |
|---|---|
| Amniotic hCG | Yes |
| Serum hCG | Yes |
| Urinary hCG | No |
| hCG from trophoblast cultures | Yes |
| Amniotic PP14 | Yes |
| Serum PP14 | Yes |
| Urinary PP14 | Yes |
| Amniotic transferrin | Yes |
| Serum Transferrin | No |
| Amniotic lgG | No |
| Serum lgG | No |
| Amniotic ✓-fetoprotein | Yes |
| Serum ✓-fetoprotein | Yes |
| Urinary ✓-fetoprotein | No |

The trophoblast culture line has been purchased and not subjected to genetic engineering. Isolation is effected in analogy to the production of gestation proteins of Examples 1 to 3.

### EXAMPLE 5

### Preparation and Testing of Glycoliposomes Containing EDTA

### EXAMPLE 5a

Phosphatidylcholine (PC; 7.44 mg), Sialyl-Lewis^{x}-polyethylene glycol(2000)-distearylphosphoethanolamine (sLe^{x}-PEG2000-DSPE; 1.26 mg), and dimyristoyl phosphatidylethanolamine (DMPE; 0.22 mg) are mixed as a chloroform solution, the solvent is removed on a rotary evaporator, and the lipid film obtained, subsequent to thorough drying, is resuspended in 1 ml of EDTA solution. Following intense agitation for several hours, multilayered vesicles (MLV) are obtained which can be put to use after several washings with phosphate-buffered isotonic saline solution (PBS; pH 7.4) and subsequent centrifugation.

### EXAMPLE 5b

To produce single-layer vesicles (SUV), the MLV from Example 5a are sonicated until a homogeneous solution with a mean vesicle diameter of about 100 nm is reached. Following centrifugation (16,000 g; 10 min), the supernatant including the liposomes is removed and put to use. Excess EDTA is removed by gel chromatography (Sephadex G50), the subsequent determination of size and content is effected as in Example 4a, furnishing a liposome population with diameters of 85 nm (PI 0.2).

### EXAMPLE 5c

To produce single-layer vesicles (LUVET), the MLV from Example 5a are extruded repeatedly in a suitable fashion (e.g. using a LiposoFast Extruder, through two polycarbonate filters with a pore diameter of 100 nm) until a homogeneous solution with a mean vesicle diameter of about 100 nm is reached. Excess EDTA is removed by gel chromatography (Sephadex G50). The content of PC and PE is determined using HPTLC. The size determination by means of quasi-elastic light scattering measurement furnishes a diameter of 114 nm (Pl 0.02). The content of liposomal PC is about 85% of the MLV suspension employed.

### EXAMPLE 5d

### Performing Adhesion Inhibition Tests Using EDTA-Glycoliposomes According to Example 5c

E-selectin (50 μl, at 5 μg/ml in Tris/calcium-containing buffer) immobilized on a microtiter plate is added with the liposomes of Example 5c, subsequently added with 100,000 51-chromium-labelled MT3 breast cancer cells per well and incubated for 1 hour at 4° C. Unbound cells are washed off, and, following lysis with NaOH, the number of bound cells is quantified via radioactivity measurement. The inhibition of tumor cell binding is 95.6%. Thus, inhibition is increased by 64% compared to liposomes of same composition but with no EDTA.

### EXAMPLE 5e

1×10⁵ HUVEC cells are stimulated with TNF-α and, after 4 hours at 37° C., added with the liposomes from Example 5c. Subsequently, 100,000 51-chromium-labelled MT3 breast cancer cells per well are added. After 1 hour at 4° C., unbound cells are washed off, and, following lysis with NaOH, the number of bound cells is quantified via radioactivity measurement. The inhibition of tumor cell binding is 61.6%. Thus, inhibition is increased by 37.8% compared to liposomes of same composition but with no EDTA.

### EXAMPLE 6

### Mimicry Molecules

Preparation of sLe^{x}-Imitating Human Recombinant Antibody Fragments from Antibody Gene Libraries Using the Phage Display Technique

Two different synthetic antibody gene libraries were used, which represent human single-chain antibody fragments (scFv). One antibody gene library consists of more than 10¹⁰ phages with different combinations of variable regions of heavy and light chains of human antibodies, in part with randomized hypervariable regions, which are linked by a peptide fragment (linker) and covalently bound to a phage coat protein (pill). It is derived from another antibody gene library (Griffiths, A. et al., 1994, EMBO J. 13, 3245-3260). The second, smaller, gene library consists of scFv preselected for active folding of the antibody fragments. The first library was provided by the laboratory of Dr. G. Winter and the second by the laboratory of Dr. I. Tomlinson (both MRC Centre for Protein Engineering, Cambridge, UK). The specific phages were selected in 2-3 runs (phage panning) using the method of proteolytic selection with KM13 helper phage (Kristensen, P. and Winters, G., Folding & Design 3, 321, 1998). The purified sLe^{x}-specific mouse antibody CSLEX1 was used as antigen (Becton Dickinson). 3 μg of the antibody was bound to 200 μl of anti-mouse IgG Dynabeads (Deutsche Dynal, Hamburg) at 4° C. over-night and subsequently for 1 hour at RT. The washed beads were subsequently blocked with 30% FCS in cell culture medium for 1 hour at RT and incubated with 5×10¹² phages from the antibody libraries for 2.5 hours at RT. Following stringent washing steps (up to 20 times PBS/0.1% Tween20 and subsequently 20 times PBS), the scFv phages binding to the binding site of the antibody were subjected to specific elution using 100 μg/ml sLe^{x}-polyacrylamide conjugates (Synthesome) and subsequently treated with trypsin (method of proteolytic selection). Alternatively, the scFv phages were eluted directly by trypsin treatment, with no specific elution by sLe^{x} carbohydrates (method of proteolytic selection). Between the selection runs, the eluted phages were grown in the bacteria using helper phages and re-selected. 2 to 3 selection runs were carried out.

Identification of Peptides Using a Peptide Gene Library Imitating sLe^{x}

In analogy to the example of generating scFv antibody fragments, specifically binding peptides were obtained in several selection runs from various own peptide gene libraries representing randomized peptides of varying length (7-12 amino acids; with and without flanking or multiple internal cysteines enabling cyclization of the peptides via sulfur bridges; Oligino, L. et al., J. Biol. Chem. 272, 29046, 1997) and having 10⁶ to 10⁸ various short peptides coupled to the phage coat protein pill. Compared to linear peptides, cyclic peptides are known to have higher stability and, in part, higher affinity. Selection and testing were performed as in the generation of the sialyl-Lewis x-imitating scFv using 3 selection runs.

Specificity Tests of the Mimicry Peptides and Mimicry scFv

The selected peptides and antibody fragments were tested in ELISA tests for binding to various sLe^{x}-specific antibodies and E-selectin, and to other IgM and IgG antibodies for control. To this end, phage-coupled forms of the peptides and antibody fragments previously purified by polyethylene glycol precipitation in 96-well plates were used. The potential mimicry peptides and mimicry scFv were examined for specific inhibition of binding of sLe^{x}-specific antibodies to sialyl-Lewis x polyacrylamide in ELISA inhibition tests. The sialyl-Lewis x polyacrylamide (0.5 μg/well) was immobilized on ELISA plates by drying, and binding of the monoclonal antibodies by the mimicry peptides or mimicry scFv in the form of synthesized peptides or purified scFv alone or coupled to phages was inhibited in a concentration-dependent fashion.

### EXAMPLE 7

Inhibition of Binding of Tumor Cells to E-Selectin by Mimicry Peptides and Mimicry scFv

E-selectin (50 μl, at 5 μg/ml in Tris/calcium-containing buffer) immobilized on a microtiter plate is added with bacteriophages having the mimicry peptides of Example 5 in the form of fusion proteins with phage coat protein pVIII at a high, not precisely defined number of copies on the surface thereof, and subsequently with 100,000 51-chromium-labelled MT3 breast cancer cells per well and incubated for 1 hour at 4° C. Unbound cells are washed off, and, following lysis with NaOH, the number of bound cells is quantified via radioactivity measurement. In a similar fashion as in the tests with glycoliposomes, inhibition of tumor cell binding is nearly complete, depending on the mimicry peptide.

The invention relates to the use of selectin-binding active agents in the form of gestation proteins or fragments thereof, of liposomes which include Ca-binding compounds, of mucin fragments obtained or derived from natural sources, or of mimicry compounds which imitate sialylated Lewis type carbohydrate structures (sLe), or combinations thereof, in the treatment and prophylaxis of diseases, in the course of which inflammatory processes are involved, such as autoimmune diseases, transplantations and arteriosclerosis. Inflammatory diseases in the meaning of the invention can be those of infectious or non-infectious nature.

1-20. (cancelled)

21. A method for inhibiting adhesion of cells from the bloodstream to activated endothelial cell tissue of blood vessels, comprising the steps of
providing selectin-binding gestation proteins or fragments thereof, of liposomes which bind to selectin and include calcium-binding compounds, of mucin fragments obtained or derived from natural sources, or of mimicry compounds which imitate sialylated Lewis type carbohydrate structures, or combinations thereof.

22. The method according to claim 21, wherein the liposomes contains EDTA.

23. The method according to claim 21, wherein the liposomes bear sialylated Lewis type carbohydrate structures as component of the liposomal membrane.

24. The method according to claim 21, wherein the liposomes bear one of selectin-binding antibodies, antibody fragments, peptides.

25. The method according to claim 24, wherein the liposomes bear selectin-binding gestation proteins.

26. The method according to claim 21, wherein the liposomes bear mimicry compounds imitating said sialylated Lewis type carbohydrate structures.

27. The method according to claim 22, wherein the liposomes bear mimicry compounds imitating said sialylated Lewis type carbohydrate structures.

28. The method according to claim 21, wherein one of gonadotropic hormones, α-fetoprotein, transferrin, glycodelins, or fragments thereof are used as gestation proteins.

29. A method for inhibiting adhesion of tumor cells or leukocytes from the bloodstream to activated endothelial cell tissue of blood vessels, comprising the steps of
providing selectin-binding gestation proteins or fragments thereof, of liposomes which bind to selectin and include calcium-binding compounds, of mucin fragments obtained or derived from natural sources, or of mimicry compounds which imitate sialylated Lewis type carbohydrate structures, or combinations thereof.

30. A method for treating and providing prophylaxis of inflammatory diseases, comprising the steps of
providing selectin-binding gestation proteins or fragments thereof, of liposomes which bind to selectin and include calcium-binding compounds, of mucin fragments obtained or derived from natural sources, or of mimicry compounds which imitate sialylated Lewis type carbohydrate structures, or combinations thereof.

31. A method for preventing partially or completely the metastasizing or providing prophylaxis of tumor diseases, comprising the steps of
providing selectin-binding gestation proteins or fragments thereof, of liposomes which bind to selectin and include calcium-binding compounds, of mucin fragments obtained or derived from natural sources, or of mimicry compounds which imitate sialylated Lewis type carbohydrate structures, or combinations thereof.

32. A pharmaceutical agent, comprising as active substance at least one selectin-binding gestation proteins or fragments thereof, and pharmaceutically conventional adjuvants.

33. The pharmaceutical agent according to claim 32, wherein the gestation proteins are one of gonadotropic hormones, α-fetoprotein, transferrin, glycodelins, or fragments thereof.

34. A pharmaceutical agent, comprising liposomes as active substance, and having the ability to bind to selectin and contain calcium-binding compounds, and pharmaceutically conventional adjuvants.

35. The pharmaceutical agent according to claim 34, wherein the liposomes includes EDTA.

36. The pharmaceutical agent according to claim 34, wherein the liposomes bear sialylated Lewis type carbohydrate structures as component of the liposomal membrane.

37. The pharmaceutical agent according to claim 35, wherein the liposomes bear sialylated Lewis type carbohydrate structures as component of the liposomal membrane

38. The pharmaceutical agent according to claim 34, wherein the liposomes bear selectin-binding antibodies, antibody fragments, peptides or other proteins.

39. The pharmaceutical agent according to claim 35, wherein the liposomes bear selectin-binding antibodies, antibody fragments, peptides or other proteins.

40. The pharmaceutical agent according to claim 38, wherein the liposomes bear selectin-binding gestation proteins.

41. The pharmaceutical agent according to claim 34, wherein the liposomes bear mimicry compounds imitating sialylated Lewis type carbohydrate structures.

42. The pharmaceutical agent according to claim 35, wherein the liposomes bear mimicry compounds imitating sialylated Lewis type carbohydrate structures.

43. A pharmaceutical agent, comprising as active substance at least one fragment of mucins obtained or derived from native sources, and pharmaceutically conventional adjuvants.

44. A pharmaceutical agent, comprising as active substance at least one mimicry compounds which imitate sialylated Lewis type carbohydrate structures, and pharmaceutically conventional adjuvants.
